(19) United States
(12) Patent Application Publication
(10) Pub. No.: US 2022/0183597 A1
(43) Pub. Date: Jun. 16, 2022

(54) IMPLANTABLE ELECTROCHEMICAL SENSORS FOR THE PH MEASUREMENT

(71) Applicants: Fabiana Arduini, Rome (RM) (IT); M.G.A. MEDICAL SRL, Rome (RM) (IT)

(72) Inventors: Fabiana Arduini, Rome (RM) (IT); Luca Fiore, Ceprano (FR) (IT); Vincenzo Mazzaracchio, Rome (RM) (IT); Alessandro Riparbelli, Rome (RM) (IT); Danila Moscone, Fondi (LT) (IT)

(73) Assignees: M.G.A. MEDICAL SRL, Rome (RM) (IT); Fabiana Arduini, Rome (RM) (IT)

(21) Appl. No.: 17/595,388

(22) PCT Filed: May 18, 2020
(86) PCT No.: PCT/EP2020/063786

§ 371 Date: Nov. 16, 2021

(30) Foreign Application Priority Data
May 20, 2019 (IT) 102019000006983

(51) Int. Cl. A61B 5/145; A61B 5/1473; A61B 5/00

(52) U.S. Cl. CPC A61B 5/14539; A61B 5/686; A61B 5/4851; A61B 5/1473

(57) ABSTRACT

Implantable electrochemical sensors for the pH measurement are herein described, comprising a working electrode and a reference electrode, in which both the working electrode and the reference electrode are integral part of an implantable orthopaedic medical device, as well as a method for their preparation and their use for monitoring the progression of bacterial infections in the implant sites of such orthopaedic medical devices.

### FIELD OF THE INVENTION

The present invention relates to an implantable electrochemical sensor for the pH measurement comprising a working electrode and a reference electrode, in which both the working electrode and the reference electrode are integral part of an implantable orthopaedic medical device.

It also relates to a method for preparing such sensor and to an implantable orthopaedic medical device comprising such electrochemical sensor.

### BACKGROUND OF THE INVENTION

The degenerative diseases (for example arthrosis) and muscle skeletal traumatic events (for example fractures) are among the most frequent traumatic chronic diseases.

Such diseases are on the rise during the last decades and further raise with the increase in the age of the patients.

Prosthetic infections represent the most frequent problem during the surgery related to articular prosthesis implant and grafts for osteosynthesis in the reduction and fixation of fractures.

Such infections are frequent in about 1-2% of articular replacement cases using osteosynthesis material during surgery

Treatment of such infections often implies prolonged hospitalization, the use of antibiotics, and additional surgical interventions.

It is evident that all these cure have an impact both on the patient prognosis and on the sanitary costs.

With regard to the first, it has been observed that in elderly patient the mortality with prosthesis infection equals to 5-10%.

Treatment of these infections requires the presence of orthopaedic surgeon and infective disease specialists.

Single examination does not provide enough information for effective resolution of the infective process.

In fact, if the blood inflammation (PCR and VES) indexes confirm their usefulness in recent literature, it is known that their clinical meaning can be unspecific and often altered due to processes deriving from different origin.

Even more controversial is the role of scintigraphic examinations. Their use is optional and correlated to the patient clinical evaluation.

Certainly scintigraphy of labelled autologous leukocytes, which represents the “gold standard” in this type of disease presents a high percentage of false positives during the six months following the surgical intervention, and this is not compatible with the need to shorten re-implantation time period.

Even though pain may not be considered a pathognomonic symptom of prosthetic infection, this symptom is considered predictive index for the treatment of early infectious complications.

In the last few years some studies about the pH changes, in tissues in which bacterial infectious processes occur, have been started.

In solution pH measurement is carried out using reagents, colorimetric strips, and glass-based or printed electrodes.

For measurements in vivo, directly in the human body, because of the fragility of glass electrodes and the difficulty of producing miniaturized device, these electrodes are not easily applicable.

For these reasons, recently some miniaturised devices have been developed for pH analysis directly in the patient body.

Devices for pH measurement, useful for monitoring the extent of infection in a wound, are known in the art.

In IEEE SENSORS Journal, Vol. 18, NO. 3, Feb. 1, 2018, 1265, an implantable sensor “wireless and battery free”, for measurement of the temperature to be inserted in the site of an orthopaedic prosthesis is described.

In EXPERT Review OF MEDICAL DEVICES 2018 (HTTPS:[ldoi.orgl]0.108011743-1-140.2018.1454310) many implantable sensors wireless, to be inserted in the site of an orthopaedic prosthesis; are described.

In WO2017041386 an apparatus and a method for monitoring the extent of infection in a wound through the measurement of the pH using multimodal pH-sensitive optical fibers and membranes is described.

In KR20170113930 (A) an apparatus for pH measurement on the skin and the transmission of the signal to an external device is described.

In CN205007119 (U) a device for measuring pH, useful for monitoring bacterial infections in wounds, is described.

In U.S. Pat. No. 7,044,982 a patch useful for repairing defects in the body is described.

In U.S. Pat. No. 7,402,172 a therapeutic intravasal patch, which facilitates a catheter insertion, is described.

### DESCRIPTION OF THE FIGURES

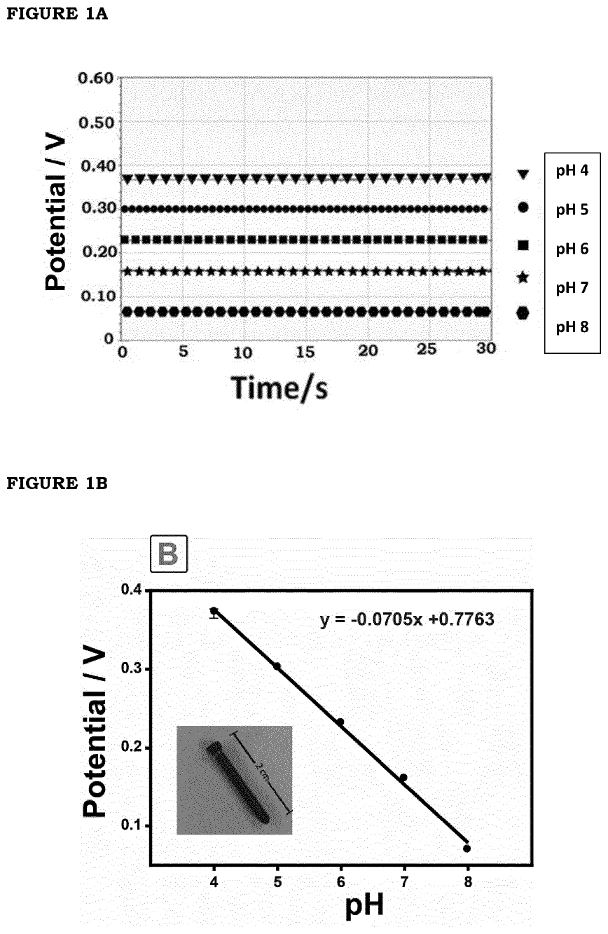

In FIG. 1A are shown the potentiometric measurements carried out in standard solutions with different pH (4, 5, 6, 7, 8), using a functionalized titanium screw according to the invention (FIG. 1B, inset).

These results showed a response to different pH values with linearity described by equation y=0.67-0.074×(R2=0.9971)

In FIG. 1B are shown:
the potential change increasing pH, these changes are described by the calibration curve; and
in the inset a titanium screw (of 2 cm) to be implanted in the body of the patient for the pH measurement, in which screw was functionalized by using iridium oxide to create a layer sensitive to H⁺ ions.

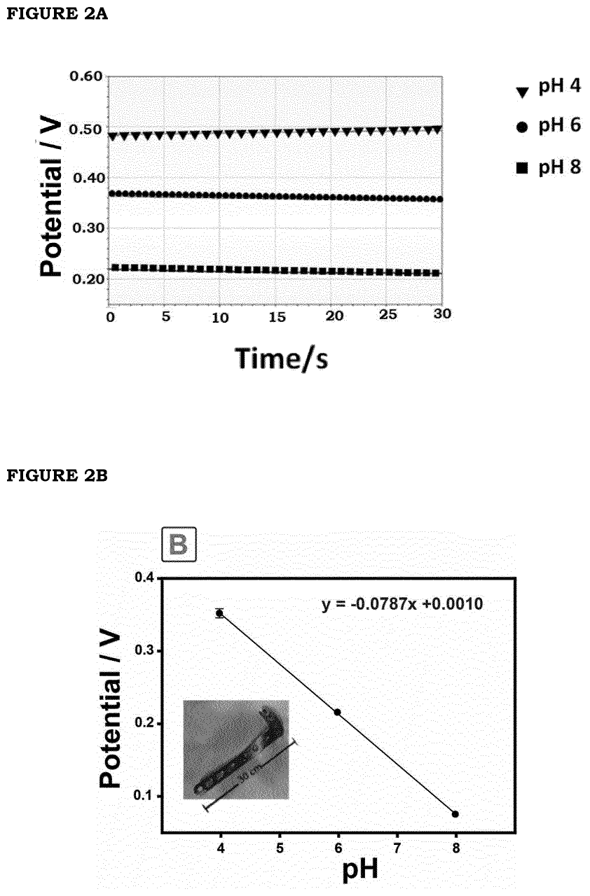

In FIG. 2A the potentiometric measurements carried out in standard solutions at different pH values (4, 6, 8), by using a titanium plate functionalized shown in inset 2B, are reported. The results related to said measurements showed a response to the different pH with a linearity described by the following equation y=0.77−0.0705x.

In FIG. 2B are shown:
the potential change in function of the increase in pH, described by calibration curve;

and
in the inset the electrode, that is a titanium plate (30 cm) usually used as system to reduce and fix femurs fracture.

This plate was functionalized with iridium oxide to create a layer sensitive to ions H⁺ for pH measurement;

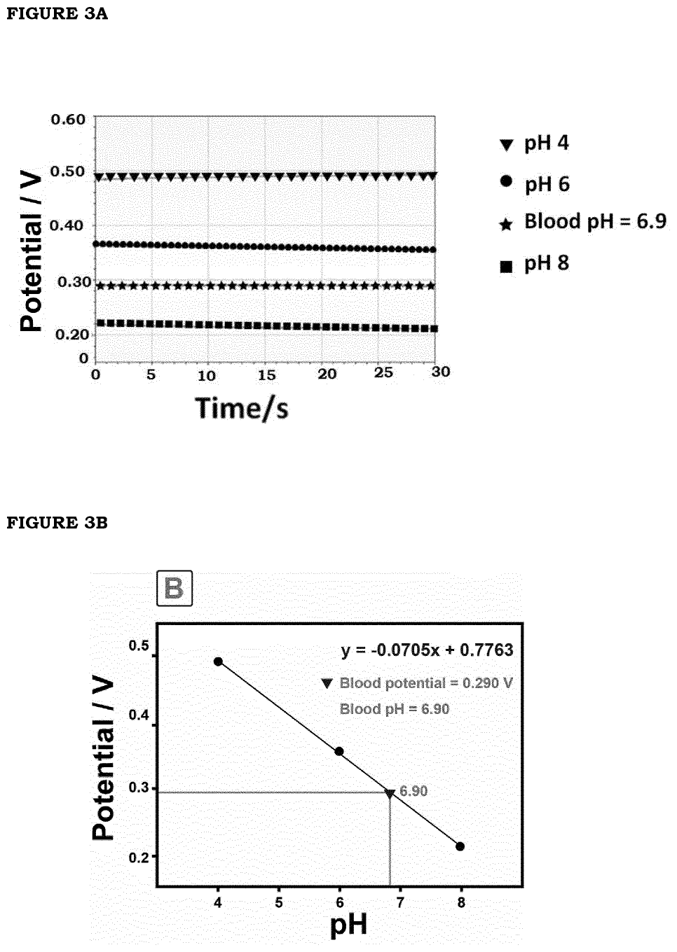

In FIG. 3A potentiometric measurements in standard solutions with different pH (4, 6, 8) and in human blood, using the functionalized titanium plate (FIG. 2B), are reported.

As shown in FIG. 3B by the interpolation of the potential value obtained was possible to calculate a pH value of 6.9 in blood.

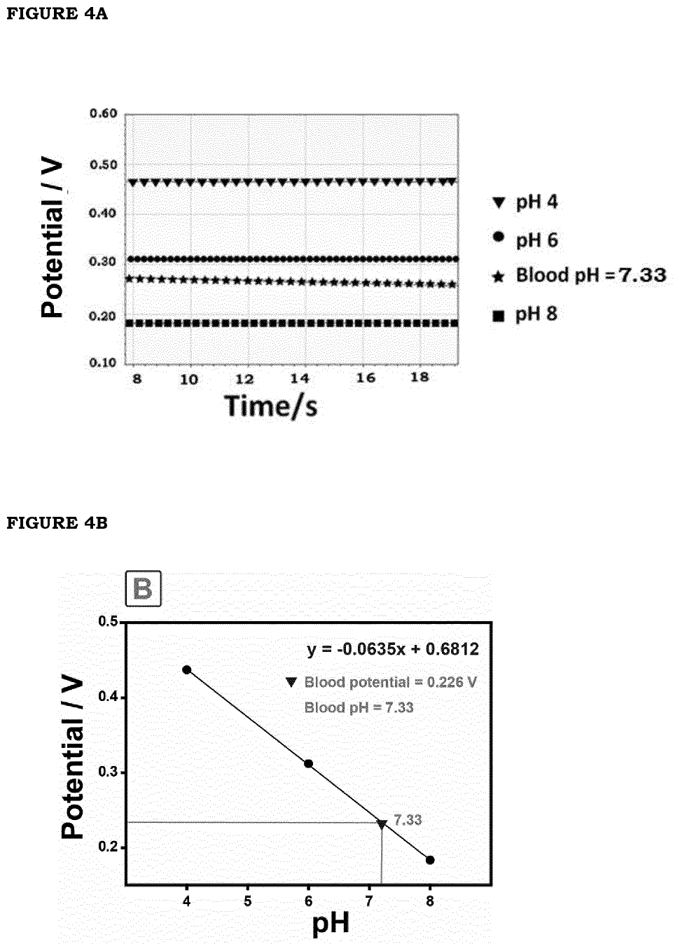

In FIG. 4A pH measurements in standard solutions with pH 4-6-8 by using the patch (the printed electrode shown in FIG. 4B inset) are reported.

In FIG. 4B inset the patch (a functionalized polyester support) used for pH measurement is reported. The calibration curve using this patch was described by the following equation y=−0.0635x+0.6812.

By interpolating the value of the potential obtained using this functionalized patch, a pH value equal to 7.33 was calculated.

### DESCRIPTION OF THE INVENTION

The present invention relates to implantable electrochemical sensors for the pH measurement comprising a working electrode and a reference electrode, in which both the working electrode and the reference electrode are integral part of an implantable orthopaedic medical device, a method for their preparation and their use for monitoring the progression of bacterial infections in the implant sites of the orthopaedic medical devices.

According to an embodiment of the invention, the implantable orthopaedic medical device is selected between a prosthesis for total or partial replacement of an articulation and means for osteosynthesis, such as, for example, Schanz screws, bars, clamps, nails, washers, guide wire, shafts, bolts, staples and plates.

According to a further embodiment of the invention, the working electrode consists in one or more patches to be applied on the implantable orthopaedic medical device.

According to another embodiment of the invention, the reference electrode consists in:
one or more patches to be applied on the implantable orthopaedic medical device;

or
a metal wire inserted within or on the implantable orthopaedic medical device, wherein, optionally, the metal is a conductive metal selected from the group comprising gold, silver, titanium, iron, steel, platinum or copper.

It is another object of the present invention an implantable orthopaedic medical device comprising the electrochemical sensor above mentioned, in which, for example, the working electrode and the reference electrode are obtained by treating the device respectively with iridium oxide or polyaniline and with a conductive metal;

wherein such treatment (or “functionalization”) is carried out:
on the surface of the device;
on the surface of at least one delimited zone of the device, zone characterised by any shape or size;
on the surface of at least a patch integrated on the medical device, in which the patch can have any shape or size;
on the surface of at least a patch integrated on the medical device, before the medical device implantation.

According to a further object of the present invention the electrochemical sensor comprises a detection and a data transmission system, provided with a battery or “battery free”, in which the variation of the signal detected by the detection and a data transmission system is transferred outside the human or animal body by means of data transmission electrical wires, or, preferably, through a wireless system using equipments known in the art, such as using “RFID” technology (radio frequency identification) for data transfer, and an external reader, using a small antenna to maximize the efficiency of the data collection and transmission.

This system is capable of working in “passive RFID” mode or battery free, by receiving the energy required for the data transfer directly from reading system, with a limited reading distance (up to 10-15 m). In “active RFID” mode, if provided with battery allows data storage in a chip, and permits higher transmissions distances (Amendola, S., 86 Marrocco, G. (2017). Optimal performance of epidermal antennas for UHF radio frequency identification and sensing. IEEE Transactions on Antennas and Propagation, 65 (2), 473-481); (Caccami, M. C., Hogan, M. P., Alfredsson, M., Marrocco, G., 86 Batchelor, J. C. (2018). A tightly Integrated Multilayer Battery antenna for RFID Epidermal Applications. IEEE Transactions on Antennas and Propagation, 66 (2), 609-617).

The implantable orthopaedic medical device according to the invention, can have the shape and dimensions of a prosthesis or of a means for osteosynthesis, obtained using a material selected from the group comprising titanium, stainless steel, chromium, cobalt, chromium-cobalt, a ceramic mixture, a conductive polymeric material, a carbon-based materials a non conductive polymeric material, such as polyethylene, alloys and combinations thereof, such as any material coated with hydroxyapatite.

The patches mentioned above can have any suitable shape or dimension, can be flexible or rigid, and can be made of using a biocompatible material selected from the group comprising: polyester, polydioxanone (PDSII), polyglycolic acid, polyglactin 910 (Vicryl), polyglecaprone 25 (Monocryl), maxonon (maxolone), linen, silk, polyamides (nylon), polyester (Novafil), polypropylene (prolene); and can be applied on the prosthesis with screws, micro-screws, glue or other bio-compatible means known in the art.

According to the invention, the implantable orthopaedic medical device comprise:
a prosthesis functionalized on its surface;
a prosthesis functionalized on its surface using iridium oxide or polyaniline; and (another area functionalized using) a conductive metal;
a prosthesis functionalized on at least a part (an area) well delimited of its surface;
a prosthesis functionalized on at least a part (an area) well delimited of its surface using iridium oxide or polyaniline; and in a different zone a conductive metal;
a prosthesis that has been functionalized by applying “on” or “in” it at least a functionalized patch;
a prosthesis that has been functionalized by applying “on” or “in” it at least a patch functionalized using iridium oxide or polyaniline and a conductive metal;
a prosthesis that has been functionalized by applying “on” or “in” it at least a patch functionalized using iridium oxide or polyaniline and a conductive metal line applied “on” or “in” the prosthesis;

and/or any other possible combination thereof.

It is a further object of the present invention a prosthesis functionalized as above reported.

It is a specific object of the present invention an implantable electrochemical sensor comprising:
at least a group of functionalized electrodes, wherein this electrodes group comprises at least a working electrode and at least a reference electrode;

characterized in that:
the working electrode is functionalized by electrochemical deposition in cyclic voltammetry by using iridium oxide or polyaniline.

As non limiting example by immersing the prosthesis, one part of the prosthesis or the patch to be applied to the prosthesis in a solution containing IrCI₄-XH₂O at a concentration between 0.01% (P/V) and 0.30% (P/V); preferably between 0.10% (P/V) and 0.20% (P/V); most preferably 0.15% (P/V) or aniline a concentration between 0.01% (P/V) and 1% (P/V); preferably between 0.05% (P/V) and 0.20% (P/V); most preferably 0.10% (P/V);

or

by immersing the prosthesis, one part of the prosthesis or the patch to be applied to the prosthesis in a solution containing aniline (electrochemical deposition) at a concentration between 0.01% (P/V) and 1% (P/V); preferably between 0.05% (P/V) and 0.20% (P/V); most preferably 0.10% (P/V).
the reference electrode is positioned/printed/linked on prosthesis or materials for osteosynthesis, as non limiting example by electrochemical deposition of a conductive ink containing a metal selected from the group comprising gold, silver, titanium, iron and steel, platinum or copper;

or by integration on/in the orthopaedic medical device of a line obtained using a conductive metal cited above; or by implantation inside/on the surface of the orthopaedic medical device of a patch functionalized with an ink containing a conductive metal above cited.

It is a further object of the present invention a process for the preparation of an implantable electrochemical sensor comprising at least a working electrode and at least one reference electrode, in which the process is characterized by the following steps:

First Step

Functionalization of the Working Electrode
the working electrode is functionalized by electrochemical deposition in cyclic voltammetry by using:

(A) iridium oxide, as non limiting example by immersing the prosthesis, one part of the prosthesis or the patch to be applied to the prosthesis in a solution containing IrCI₄-XH₂O at a concentration between 0.01% (P/V) and 0.30% (P/V); preferably between 0.10% (P/V) and 0.20% (P/V); most preferably 0.15% (P/V) or aniline a concentration between 0.01% (P/V) and 1% (P/V); preferably between 0.05% (P/V) and 0.20% (P/V); most preferably 0.10% (P/V); following the procedure described in Ges et Al. (Ges, I. A., Ivanov, B. L., Schaffer, D. K., Lima, E. A., Werdich, A. A., 86 Baudenbacher, F. J. (2005). Thin-film IrOx pH microelectrode for microfluidic-based microsystems. Biosensors and Bioelectronics, 21(2), 248-256);

or

(B) by immersing the prosthesis, one part of the prosthesis or the patch to be applied to the prosthesis in a solution containing aniline at a concentration between 0.01% (P/V) and 1% (P/V); preferably between 0.05% (P/V) and 0.20% (P/V); most preferably 0.10% (P/V) (Gao, W., & Song, J. (2009). Polyaniline film based amperometric pH sensor using a novel electrochemical measurement system. Electroanalysis 21(8), 973-978; Chinnathambi, S., 86 Euverink, G. J. W. (2018). Polyaniline functionalized electrochemically reduced graphene oxide chemiresistive sensor to monitor the pH in real time during microbial fermentations. Sensors and Actuators B: Chemical, 264, 38-44);

subsequently the method of electrochemical deposition in cyclic voltammetry on the surface of the orthopaedic medical device a layer/layer sensitive to ions H⁺ may be performed.

Second Step

Reference Electrode Functionalization
as non limiting example, the reference electrode is functionalized/printed on the prosthesis or materials for osteosynthesis by printing an ink comprising a conductive metal selected from the group comprising platinum, gold, silver or copper;
alternatively the reference electrode can be constituted by a patch to be applied to the device of the invention, in which said patch is functionalized by using an ink comprising a conductive metal,

or
the reference electrode can be constituted by a metal wire implanted within or on the surface of the orthopaedic medical device according to the invention, in which the wire is obtained by using a conductive metal above mentioned.

How to print an ink containing a metal on a solid surface, or how to apply on/in a prosthesis or means for osteosynthesis a patch or a metal wire, are procedures well known in the state of art.

The orthopaedic medical device according to the invention, can be provided with a suitable battery or preferably is of the type battery free.

The variation of the signal detected by the sensor according to the invention can be transmitted/transferred outside of the human or animal body by means of data transmission electrical wires or, preferably, by using a wireless systems mentioned above, using the data transmission technology “RFID”.

It is evident to a skilled in the state of art that on the implantable orthopaedic medical device according to the invention may be present more measurement points (more sensors) for pH monitoring.

It is also evident to the skilled in in the state of the art that to date the wireless systems mentioned above, and further systems known in the art, are suitable of transmitting and managing multiple measurements.

The following Examples illustrate the invention without limiting it and by making reference to the Figures described above.

### EXAMPLES

### Example 1

Preparation of a Titanium Orthopaedic Medical Device, Named “Screw”, Implantable in Human or Animal Body, Functionalized for pH Measurement

An implantable titanium screw (FIG. 1B inset) was used.

The surface of the screw was functionalized (externally) by electrochemical deposition in cyclic voltammetry, by using iridium oxide. The screw was immersed in a solution containing IrCI₄-XH₂O following the procedure described in Ges et Al. (Ges, I. A., Ivanov, B. L., Schaffer, D. K., Lima, E. A., Werdich, A. A., & Baudenbacher, F. J. (2005). Thin-film IrOx pH microelectrode for microfluidic-based microsystems. Biosensors and Bioelectronics, 21(2), 248-256).

Using the method of electrochemical deposition in cyclic voltammetry on the surface of the screw, a layer sensitive to H⁺ was obtained.

The screw thus obtained was used to measure the pH in standard solutions at pH 4, 5, 6, 7 and 8.

In FIG. 1A shows the potentiometric measurements in standard solutions with different pH values, using such screw.

In FIG. 1B shows the corresponding calibration curve.

The measurements made using this functionalized screw showed a response to the different pH with a linearity described by equation y=0.67−0.074×(R2=0.9971) (FIG. 1B).

### Example 2

Process for the Preparation of a Titanium Orthopaedic Medical Device, Named “Plate”, Implantable in Human or Animal Body Functionalized for pH Measurement

For the preparation of the titanium implantable device, a titanium plate (FIG. 2B inset) was used, this titanium plate usually is used as orthopaedic medical device for the joint reconstruction of femurs.

The surface of the plaque was functionalized as described in Example 1.

The titanium plate electrode thus obtained for pH was used to measure pH in standard solutions at pH 4-6-8.

In FIG. 2A are shown the potentiometric measurements in standard solutions at different pH values

In FIG. 2B is shown the corresponding calibration curve.

The equation of the calibration curve gave results equal to y=0.77−0.0705x (FIG. 2-B).

### Example 3

pH measurement in an unknown blood sample, by using the plate of

### Example 2

The titanium plate of Example 2 was used to perform the measurement of pH in an unknown sample of human blood.

As shown in FIG. 3, by interpolating the value of potential obtained with the electrode according to the invention, it was possible to calculate a pH value equal to 6.9.

This value was confirmed by the measurement carried out by a reference method, using a classic glass laboratory electrode.

### Example 4

Process for the Preparation of a Patch to be Integrated in an Orthopaedic Medical Device Implantable in Human or Animal Body

For the preparation of the patch, a polyester support was used (FIG. 4B inset).

The surface of the support was functionalized as described in Example 1.

The patch obtained was used to measure the pH in standard solutions at pH 4-6-8,

The equation of the calibration curve gave results equal to y=−0.0635x+0.6812 (FIG. 4B).

In FIG. 4A the potentiometric measurements of standard solutions with different pH values are shown; while in FIG. 4B the calibration curve is shown.

As shown in FIG. 4B, by interpolating the value of potential obtained with the electrode according to the invention a pH value equal to 7.33 was calculated.

This value was confirmed (pH 7.44) by using as reference method a glass laboratory electrode.

1. An implantable electrochemical sensor for the pH measurement comprising a working electrode and a reference electrode, wherein both the working electrode and the reference electrode are integral part of an implantable orthopaedic medical device.

2. The implantable electrochemical sensor of claim 1, wherein the implantable orthopaedic medical device is selected between a prosthesis for total or partial replacement of an articulation and means for osteosynthesis.

3. The implantable electrochemical sensor of claim 1, wherein the working electrode consists in one or more patches to be applied on the implantable orthopaedic medical device.

4. The implantable electrochemical sensor of claim 1, wherein the reference electrode consists of
one or more patches to be applied on the implantable orthopaedic medical device; or
a metal wire inserted within or on the implantable orthopaedic medical device.

5. The implantable electrochemical sensor of claim 4, wherein the metal is a conductive metal selected from the group consisting of gold, silver, titanium, iron, steel, platinum and copper.

6. A method for preparing the implantable electrochemical sensor of claim 1, wherein
the working electrode is prepared by
(A) totally or partially immersing the implantable orthopaedic medical device, or one or more patches to be applied on or in the same, in a solution containing IrCI₄-XH₂) at a concentration between 0.01% (P/V) and 0.30% (P/V);
or
(B) totally or partially immersing the implantable orthopaedic medical device, or one or more patches to be applied on or in the same, in a solution containing aniline at a concentration between 0.01% (P/V) and 1% (P/V); and
the reference electrode is prepared by
totally or partially applying on the implantable orthopaedic medical device, or on one or more patches to be put on or in the same, an ink comprising a conductive metal selected from the group consisting of platinum, gold, silver and copper.

7. The method of claim 6, wherein the patch is prepared by using a biocompatible material selected from the group consisting of: polyester, polydioxanone, polyglycolic acid, polyglactin 910, poliglecaprone 25, maxonon, flax, silk, polyamides and polypropylene.

8. An implantable orthopaedic medical device comprising the electrochemical sensor of claim 1.

9. The implantable orthopaedic medical device of claim 8, comprising a detection and transmission data system.

10. The implantable orthopaedic medical device of claim 9, wherein the transmission data system is wireless.

11. The implantable electrochemical sensor of claim 2, wherein said mean for osteosynthesis are selected from the group consisting of Schanz screws, bars, clamps, nails, washers, guide wire, shafts, bolts, staples and plates.

12. The method of claim 6, wherein the concentration of said IrCI₄ is between 0.10% (P/V) and 0.20% (P/V).

13. The method of claim 6, wherein the concentration of said IrCI₄ is 0.15% (P/V).

14. The method of claim 6, wherein the concentration of said aniline is between 0.05% (P/V) and 0.20% (P/V).

15. The method of claim 6, wherein the concentration of said aniline is 0.10% (P/V).
